(19) United States
(12) Patent Application Publication
(10) Pub. No.: US 2017/0210286 A1
(43) Pub. Date: Jul. 27, 2017

(54) Vehicle Occupant Safety System

(71) Applicant: Nur Kasin, Eagan, MN (US)

(72) Inventor: Nur Kasin, Eagan, MN (US)

(21) Appl. No.: 15/007,421

(22) Filed: Jan. 27, 2016

(51) Int. Cl. B60Q 5/00; G01N 33/00; G01K 13/00

(52) U.S. Cl. CPC B60Q 5/005; G01K 13/00; G01N 33/004

(57) ABSTRACT

A vehicle occupant safety system includes a vehicle that may contain an occupant. A detection unit is provided. The detection unit detects the occupant when the vehicle is left unattended. The detection unit detects an interior temperature of the vehicle. The detection unit detects motion thereby facilitating the detection unit to detect the occupant. The detecting unit generates an alarm sequence when the interior temperature of the vehicle moves outside a trigger range of temperature and the detection unit detects the occupant. An alarm unit is coupled to the vehicle and the alarm unit may emit an audible alarm for an extrinsic observer. The alarm unit is in communication with the detection unit. The alarm emits an audible alarm when the detection unit generates the alarm sequence. Thus, the alarm unit notifies the extrinsic observer that the occupant is in danger.

### BACKGROUND OF THE DISCLOSURE

Field of the Disclosure

The disclosure relates to safety devices and more particularly pertains to a new safety device for inhibiting an occupant from being exposed to a life threatening temperature in an unattended vehicle.

Summary of the Disclosure

An embodiment of the disclosure meets the needs presented above by generally comprising a vehicle that may contain an occupant. A detection unit is provided. The detection unit detects the occupant when the vehicle is left unattended. The detection unit detects an interior temperature of the vehicle. The detection unit detects motion thereby facilitating the detection unit to detect the occupant. The detecting unit generates an alarm sequence when the interior temperature of the vehicle moves outside a trigger range of temperature and the detection unit detects the occupant. An alarm unit is coupled to the vehicle and the alarm unit may emit an audible alarm for an extrinsic observer. The alarm unit is in communication with the detection unit. The alarm emits an audible alarm when the detection unit generates the alarm sequence. Thus, the alarm unit notifies the extrinsic observer that the occupant is in danger.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a perspective view of an occupant safety system according to an embodiment of the disclosure.

FIG. 2 is a bottom phantom view of a first housing of an embodiment of the disclosure.

FIG. 3 is a right side view of a first housing of an embodiment of the disclosure.

FIG. 4 is a schematic view of an embodiment of the disclosure.

FIG. 5 is a perspective in-use view of an embodiment of the disclosure.

### DESCRIPTION OF THE PREFERRED EMBODIMENT

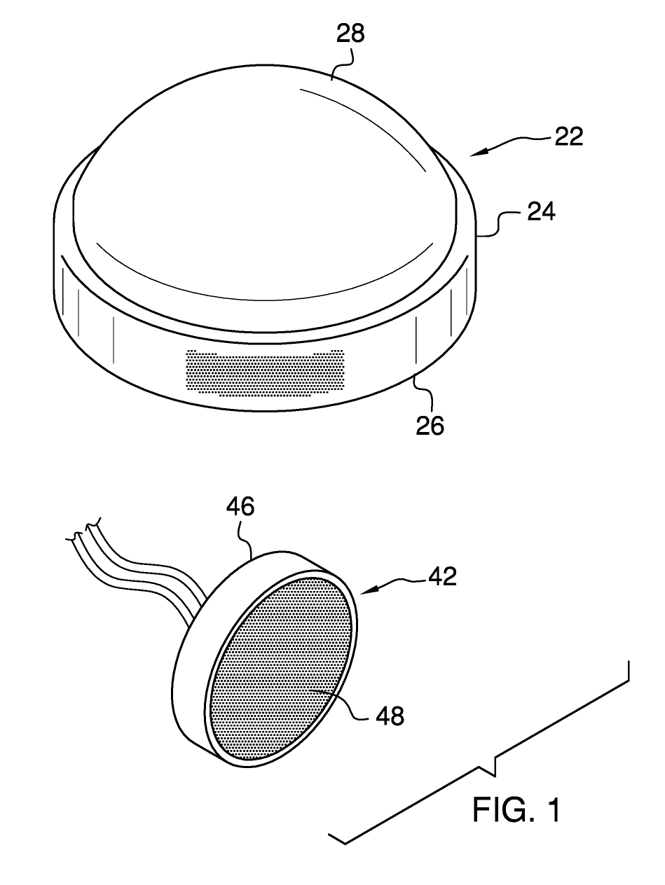

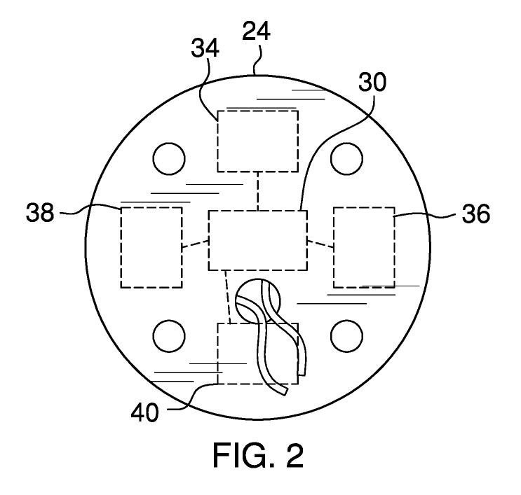

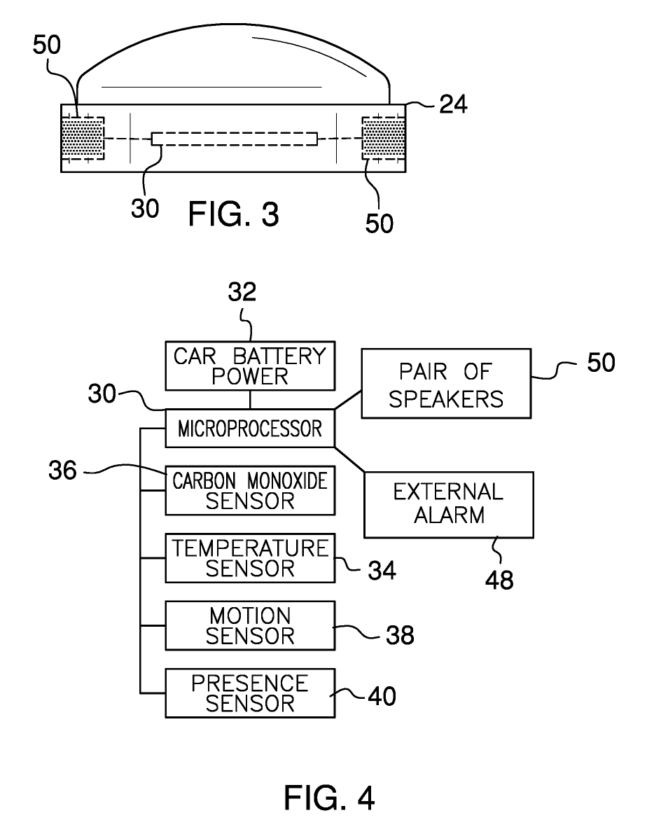

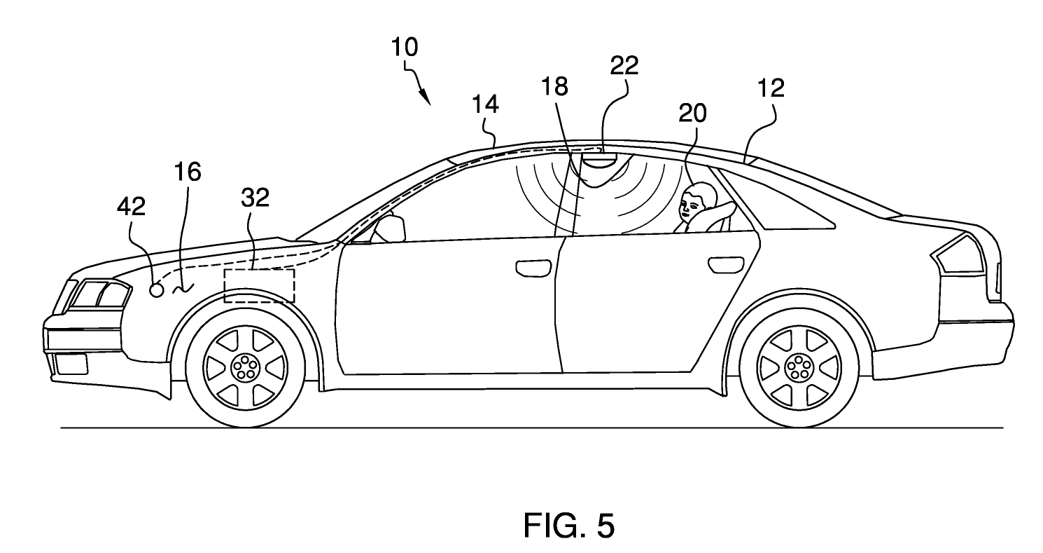

With reference now to the drawings, and in particular to FIGS. 1 through 5 thereof, a new safety device embodying the principles and concepts of an embodiment of the disclosure and generally designated by the reference numeral 10 will be described.

As best illustrated in FIGS. 1 through 5, the vehicle occupant safety system 10 generally comprises a vehicle 12. The vehicle 12 has a roof 14 and an exterior surface 16. The roof 14 has an inside surface 18 and the vehicle 12 may contain an occupant 20. The vehicle 12 may be a passenger vehicle or the like. The occupant 20 may be a child, a pet or a developmentally disabled adult.

A detection unit 22 is provided and the detection unit 22 may detect the occupant 20 when the vehicle 12 is left unattended. The detection unit 22 detects an interior temperature of the vehicle 12. The detection unit 22 detects motion thereby facilitating the detection unit 22 to detect the occupant 20. The detecting unit 22 generates an alarm sequence when the interior temperature of the vehicle 12 drops above a maximum trigger temperature and the detection unit 22 detects the occupant 20. The detection unit 22 generates the alarm sequence when the interior temperature of the vehicle 12 exceeds a maximum trigger temperature and the detection unit 22 detects the occupant 20.

The detection unit 22 comprises a first housing 24 that has a top side 26 and a bottom side 28. The top side 26 is coupled to the inside surface 18 of the roof 14. The bottom side 28 may be concavely arcuate with respect to the top side 26. Thus, the bottom side 28 may define a dome. A processor 30 is positioned within the first housing 24. The processor 30 is electrically coupled to an electrical system 32 of the vehicle 12. The processor 30 may comprise an electronic processor or the like.

A temperature sensor 34 is coupled to the first housing 24 and the temperature sensor 34 detects the interior temperature of the vehicle 12. The temperature sensor 34 is electrically coupled to the processor 30. The processor 30 generates a first condition when the interior temperature drops below the minimum trigger temperature and when the interior temperature exceeds the maximum trigger temperature. The temperature sensor 34 may comprise an electronic temperature sensor or the like. The minimum trigger temperature may be a temperature ranging between approximately thirty degrees Fahrenheit and forty degrees Fahrenheit. The maximum trigger temperature may be a temperature ranging between approximately eighty degrees Fahrenheit and ninety degrees Fahrenheit.

A carbon monoxide sensor 36 is coupled to the first housing 24. The carbon monoxide sensor 36 detects a level of carbon monoxide within interior of the vehicle 12. The carbon monoxide sensor 36 is electrically coupled to the processor 30. The processor 30 generates the first condition when the carbon monoxide sensor 36 detects a trigger amount of carbon monoxide within the vehicle 12. The carbon monoxide sensor 36 may comprise an electronic carbon monoxide sensor or the like. The trigger amount of carbon monoxide may be an amount ranging between approximately seventy ppm and one hundred ppm.

A motion sensor 38 is coupled to the first housing 24 and the motion sensor 38 detects motion within the vehicle 12. The motion sensor 38 is electrically coupled to the processor 30. The processor 30 generates the alarm sequence when the motion sensor 38 detects motion and the processor 30 has generated the first condition. The motion sensor 38 may comprise an electronic motion sensor or the like.

A thermal sensor 40 is coupled to the first housing 24 and the thermal sensor 40 detects a thermal signature of the occupant 20. The thermal sensor 40 is electrically coupled to the processor 30. The processor 30 generates the alarm sequence when the thermal sensor 40 detects the thermal signature of the occupant 20 and the processor 30 has generated the first condition. The thermal sensor 40 may comprise an electronic thermal sensor or the like.

An alarm unit 42 is coupled to the vehicle 12 and the alarm unit 42 may emit an audible alarm for an extrinsic observer 44. The alarm unit 42 is in communication with the detection unit 22. The alarm unit 42 emits an audible alarm when the detection unit 22 generates the alarm sequence. Thus, the alarm unit may notify the extrinsic observer 44 that the occupant 20 is in danger thereby facilitating the extrinsic observer 44 to remove the occupant 20 from the vehicle 12.

The alarm unit 42 comprises a second housing 46 that is coupled to the exterior surface 16 of the vehicle 12. A speaker 48 is coupled to the second housing 24. Thus, the speaker 48 may emit the audible alarm for the extrinsic observer 44. The speaker 48 is electrically coupled to the processor 30 such that the speaker 48 emits the audible alarm when the processor 30 generates the alarm sequence.

The detection unit 22 may be incorporated into an existing dome light of the vehicle 12. The detection unit 22 may be a factory installed component of the vehicle 12. The detection unit 22 may be retrofitted to an existing vehicle 12. A pair of speakers 50 may each be positioned within the first housing 24. Each of the pair of speakers 50 may be electrically coupled to the processor 30. Thus, each of the pair of speakers 50 may emit the audible alarm when the processor 30 generates the alarm sequence.

In use, the occupant 20 is left in the vehicle 12 unattended. The speaker 48 emits the audible alarm when the occupant 20 is in the vehicle and the processor 30 generates the alarm sequence. Thus, the extrinsic observer 44 is notified of an emergency situation. The extrinsic observer 44 removes the occupant 20 from the vehicle 12. The extrinsic observer 44 may contact emergency responders if the extrinsic observer 44 cannot remove the occupant 20 from the vehicle 12. Thus, the occupant 20 is removed from a potentially life threatening environment.

With respect to the above description then, it is to be realized that the optimum dimensional relationships for the parts of an embodiment enabled by the disclosure, to include variations in size, materials, shape, form, function and manner of operation, system and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A vehicle occupant safety system comprising:
a vehicle being configured to contain an occupant;
a detection unit being configured to detect the occupant when the vehicle is left unattended, said detection unit being configured to detect an interior temperature of said vehicle, said detection unit being configured to detect motion thereby facilitating said detection unit to detect the occupant, said detecting unit generating an alarm sequence when the interior temperature of said vehicle drops below a minimum trigger temperature and said detection unit detects the occupant, said detection unit generating the alarm sequence when the interior temperature of said vehicle exceeds a maximum trigger temperature and said detection unit detects the occupant, wherein said detection unit comprises
a first housing having a top side and a bottom side, said top side being coupled to an inside surface of a roof, and
a processor being positioned within said first housing, said processor being electrically coupled to said vehicle;
an alarm unit being coupled to said vehicle wherein said alarm unit is configured to emit an audible alarm for an extrinsic observer, said alarm unit being in communication with said detection unit, said alarm emitting an audible alarm when said detection unit generates said alarm sequence wherein said alarm unit is configured to notify the extrinsic observer that the occupant is in danger;
a temperature sensor being coupled to said first housing wherein said temperature sensor is configured to detect the interior temperature of said vehicle, said temperature sensor being electrically coupled to said processor, said processor generating a first condition when the interior temperature drops below said minimum trigger temperature and when the interior temperature exceeds said maximum trigger temperature; and
a carbon monoxide sensor being coupled to said first housing wherein said carbon monoxide sensor is configured to detect a level of carbon monoxide within interior of said vehicle, said carbon monoxide sensor being electrically coupled to said processor, said processor generating said first condition when said carbon monoxide sensor detects a trigger amount of carbon monoxide within said vehicle.

2. (canceled)

3. (canceled)

4. (canceled)

5. The system according to claim 1, further comprising a motion sensor being coupled to said first housing wherein said motion sensor is configured to detect motion within said vehicle, said motion sensor being electrically coupled to said processor, said processor generating said alarm sequence when said motion sensor detects motion and said processor has generated said first condition.

6. The system according to claim 1, further comprising a thermal sensor being coupled to said first housing wherein said thermal sensor is configured to detect a thermal signature of the occupant, said thermal sensor being electrically coupled to said processor, said processor generating said alarm sequence when said thermal sensor detects the thermal signature of the occupant and said processor has generated said first condition.

7. The system according to claim 1, wherein:
said detection unit includes a processor; and
said alarm unit comprises:
a second housing being coupled to an exterior surface of said vehicle; and
a speaker being coupled to said second housing wherein said speaker is configured to emit the audible alarm for the extrinsic observer, said speaker being electrically coupled to said processor such that said speaker emits the audible alarm when said processor generates said alarm sequence.

8. A vehicle occupant safety system comprising:
a vehicle having a roof and an exterior surface, said roof having an inside surface, said vehicle being configured to contain a occupant;
a detection unit being configured to detect the occupant when the vehicle is left unattended, said detection unit being configured to detect an interior temperature of said vehicle, said detection unit being configured to detect motion thereby facilitating said detection unit to detect the occupant, said detecting unit generating an alarm sequence when the interior temperature of said vehicle drops below a minimum trigger temperature and said detection unit detects the occupant, said detection unit generating the alarm sequence when the interior temperature of said vehicle exceeds a maximum trigger temperature and said detection unit detects the occupant, said detection unit comprising:
a first housing having a top side and a bottom side, said top side being coupled to said inside surface of said roof,
a processor being positioned within said first housing, said processor being electrically coupled to said vehicle,
a temperature sensor being coupled to said first housing wherein said temperature sensor is configured to detect the interior temperature of said vehicle, said temperature sensor being electrically coupled to said processor, said processor generating a first condition when the interior temperature drops below said minimum trigger temperature and when the interior temperature exceeds said maximum trigger temperature,
a carbon monoxide sensor being coupled to said first housing wherein said carbon monoxide sensor is configured to detect a level of carbon monoxide within interior of said vehicle, said carbon monoxide sensor being electrically coupled to said processor, said processor generating said first condition when said carbon monoxide sensor detects a trigger amount of carbon monoxide within said vehicle,
a motion sensor being coupled to said first housing wherein said motion sensor is configured to detect motion within said vehicle, said motion sensor being electrically coupled to said processor, said processor generating said alarm sequence when said motion sensor detects motion and said processor has generated said first condition, and
a thermal sensor being coupled to said first housing wherein said thermal sensor is configured to detect a thermal signature of the occupant, said thermal sensor being electrically coupled to said processor, said processor generating said alarm sequence when said thermal sensor detects the thermal signature of the occupant and said processor has generated said first condition; and
an alarm unit being coupled to said vehicle wherein said alarm unit is configured to emit an audible alarm for an extrinsic observer, said alarm unit being in communication with said detection unit, said alarm emitting an audible alarm when said detection unit generates said alarm sequence wherein said alarm unit is configured to notify the extrinsic observer that the occupant is in danger, said alarm unit comprising:
a second housing being coupled to said exterior surface of said vehicle, and
a speaker being coupled to said second housing wherein said speaker is configured to emit the audible alarm for the extrinsic observer, said speaker being electrically coupled to said processor such that said speaker emits the audible alarm when said processor generates said alarm sequence.
